(19) United States
(12) Patent Application Publication
(10) Pub. No.: US 2024/0260571 A1
(43) Pub. Date: Aug. 8, 2024

(54) ANTI-MICROBIAL COMPOSITION

(71) Applicant: Byotrol PLC, Manchester (GB)

(72) Inventors: Huw EVANS, Ince, Chester (GB); Mark NEWTON, Ince, Chester (GB); Christopher PLUMMER, Ince, Chester (GB); Lauren Mairead BURNS, Ince, Chester (GB)

(21) Appl. No.: 18/019,651

(22) PCT Filed: Aug. 3, 2021
(86) PCT No.: PCT/GB2021/052002

§ 371 Date: Feb. 3, 2023

(30) Foreign Application Priority Data
Aug. 5, 2020 (GB) 2012191.9

(51) Int. Cl. A01N 33/12; A01P 1/00; C11D 1/72; C11D 3/48; C11D 17/04

(52) U.S. Cl. CPC A01N 33/12; A01P 1/00; C11D 1/72; C11D 3/48; C11D 17/049; C11D 2111/14

(57) ABSTRACT

Anti-microbial compositions are described which contain:
(i) an active agent component containing:
(A) a compound of formula (A)

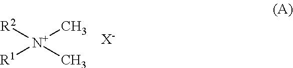

wherein R¹ and R² are independently a C₈₋₁₂ alkyl group and X⁻ is a halide anion such as chloride, bromide, fluoride, iodide or sulphonate, saccharinate, carbonate or bicarbonate;
(B) a compound of formula (B)

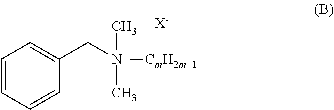

wherein m is from 8 to 18, and X⁻ is a halide anion such as chloride, bromide, fluoride, iodide or sulphonate, saccharinate, carbonate or bicarbonate; and
(C) a compound of formula (C)

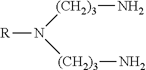

where R is an unsubstituted C₈ to C₁₈ alkyl group;
wherein:
(a) (C) is present in an amount of from 5 to 95 wt % of the active agent component; and
(b) the ratio by weight of A:B is 1:9 to 9:1; and
(ii) a polar solvent.

This invention relates to anti-microbial compositions.

Microorganisms and viruses are known to present health hazards due to infection or contamination. They can also cause spoilage of items such as clothing and unpleasant odours. For example, when micro-organisms are present on the surface of a substrate they can replicate rapidly to form colonies. Viruses are responsible for a wide range of mammalian disease which represents a great cost to society. The effects of viral infection can range from common flu symptoms to serious respiratory problems and can result in death, particularly amongst the young, elderly and immunocompromised members of the community.

Many anti-microbial agents that can destroy microorganisms and/or viruses that are present in a wide range of environments such as medical, industrial, commercial, domestic and marine environments are known. Many of the known anti-microbial agents have previously been included in compositions for use in various applications and environments.

The known anti-microbial agents and the compositions that contain these anti-microbial agents destroy microorganisms and/or viruses by a number of different mechanisms.

For example, many anti-microbial agents are poisonous to the microorganisms and/or viruses and, therefore, destroy the microorganisms and/or viruses with which they are contacted. Examples of this type of anti-microbial agent include hypochlorites (bleaches), phenol and compounds thereof, and salts of copper, tin and arsenic. However, some of these agents can be highly toxic to humans and animals as well as to microorganisms and/or viruses. Consequently these anti-microbial agents are dangerous to handle, and specialist handling, treatment and equipment are therefore required in order to handle them safely. The manufacture and disposal of compositions comprising this type of anti-microbial agent can, therefore, be problematic. There can also be problems associated with the use of compositions containing this type of anti-microbial agent, particularly in consumer materials where it is difficult to ensure that they are used for designated purposes.

Herein, unless the context indicates otherwise, “toxicity” is intended to refer to toxicity to complex organisms such as mammals. References to “toxic” are to be construed accordingly.

Other anti-microbial agents currently in use include antibiotic type compounds. Antibiotics disrupt the biochemistry within microorganisms. Although antibiotics are effective, it is believed that they may selectively permit the development of resistant strains of the species that they are used against.

Another method of microbial control is the use of oxidising agents in materials, such as household bleach, which can be based on hypochlorite or peroxides such as hydrogen peroxide. These materials are effective in a wet environment for sterilization and cleansing but stop working shortly after drying.

The listing or discussion of an apparently prior-published document in this specification should not necessarily be taken as an acknowledgment that the document is part of the state of the art or is common general knowledge.

It is desirable when formulating an anti-microbial product for it to be as effective as possible against a broad range of organisms including bacteria, yeast, fungi, and viruses. This is not easy since different organisms are not all equally hard to kill and can require different levels and different ratios of biocides to be effective. It is desirable to have a single composition which can be diluted to different extents to be effective against a broad range of organisms. The challenge is that one ratio of biocides that is effective against one organism for example bacteria is often not effective against another for example a fungus.

There is a need to provide compositions for a variety of applications and uses, such as cleaning applications that have anti-microbial properties and that address one or more of the problems set out above. In particular, there is a need to provide compositions that provide a broad spectrum anti-microbial effect that uses low concentrations of actives, and therefore avoid one or more of the problems set out above. There is also a need to provide a single composition which can be diluted to different extents to be effective against a broad range of organisms.

However, it is not a straight forward matter to address these needs. There are regulations such as the Biocidal Products Regulations (BPR Regulation (EU)528/2012) which regulates the use of anti-microbial agents both in terms of the nature and the amount of a given anti-microbial agent that may be used.

Additionally, the potential reactivity of an anti-microbial agent once in a composition is important as some anti-microbial agents are rendered inactive by chemical reaction. Even where an anti-microbial agent is not deactivated by chemical reaction it may have its activity suppressed by other components of the composition.

The present inventors have surprisingly found that the foregoing deficiencies can be overcome by certain combinations of components. It has also been found that compositions containing these combinations of components can have some surprising and unexpected properties.

In particular, the present invention provides anti-microbial compositions suitable for a variety of consumer applications. The compositions of the invention are particularly suitable for use on hard surfaces (porous and non-porous hard surfaces) and/or soft surfaces and/or other porous surfaces. Some compositions of the invention are also suitable for cleaning such surfaces, i.e. cleaning hard surfaces. Some compositions of the invention provide a residual viral effect.

The compositions of the invention can be used for a range of applications and are particularly suitable for use on hard surfaces (porous and non-porous hard surfaces) and porous such as soft surfaces. The compositions of the invention may be used for non-therapeutic treatment of hard surfaces. For example, the compositions of the invention may be used on hard surfaces indoors such as those found in a domestic setting, an office or a public building such as a hospital or out of doors. The compositions of the invention may also be used on porous surfaces. For example, the compositions of the invention may be used on non-living porous surfaces, such as upholstery, carpets, floor coverings, leather, fabrics, plastics, clothes, gloves, PPE (personal protective equipment) and/or the compositions of the invention may be used on living porous surfaces, such as human skin and/or hair, or animal fur and/or skin. For example, domestic animals, such as cats, dogs, guinea pigs, and other animals, such as horses, sheep, cows and other livestock.

As used herein, by the term cleaning we mean the removal of soils, such as dirt, soap scum and limescale.

The present invention provides an anti-microbial composition comprising:
(i) an active agent component comprising:
(A) a compound of formula (A)

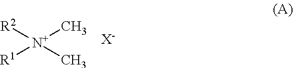

wherein R¹ and R² are independently a C₈₋₁₂ alkyl group and X⁻ is a halide anion such as chloride, bromide, fluoride, iodide or sulphonate, saccharinate, carbonate or bicarbonate;
(B) a compound of formula (B)

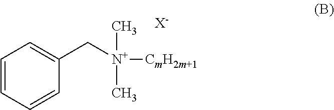

wherein m is from 8 to 18, and X⁻ is a halide anion such as chloride, bromide, fluoride, iodide or sulphonate, saccharinate, carbonate or bicarbonate; and
(C) a compound of formula (C)

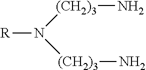

where R is an unsubstituted C₈ to C₁₈ alkyl group;
wherein:
(a) (C) is present in an amount of from 5 to 95 wt % of the active agent component; and
(b) the ratio by weight of A:B is 1:9 to 9:1; and
(ii) a polar solvent.

This composition is hereinafter referred to as the composition of the invention.

While it is envisaged that compositions of the invention can contain additional ingredients as described below and other ingredients that are standard in the art, the compositions of the invention may consist of or consist essentially of the components listed in the paragraph above.

The present inventors has found that certain compositions with the ternary biocide system, composed of Compounds (A), (B) and (C) as defined above and in particular N-(3-aminopropyl)-N-dodecylpropane-1,3-diamine, di-n-decyldimethyl ammonium chloride (DDAC) and alkyl dimethylbenzylammonium chloride (benzalkonium chloride, ADBAC) can be effective against a range of organisms: bacteria, yeast, fungi and viruses when used in the ratios described herein. They have also found that by adjusting the composition of certain compositions containing this ternary biocide system anti-microbial compositions effective against different organisms can be obtained.

For the avoidance of doubt, in this specification when we use the term “comprising” or “comprises” we mean that the composition or formulation or component being described must contain the listed ingredient(s) but may optionally contain additional ingredients. When we use the term “consisting essentially of” or “consists essentially of” we mean that the composition or formulation or component being described must contain the listed ingredient(s) and may also contain small (for example up to 5% by weight, or up to 1% or 0.1% by weight) of other ingredients provided that any additional ingredients do not affect the essential properties of the composition, formulation or component. When we use the term “consisting of” we mean that the composition or formulation or component being described must contain the listed ingredient(s) only. These terms can be applied in an analogous manner to processes, methods and uses.

By “substantially free” we mean that the composition or formulation or component being described contains less than 3% by weight, preferably less than 1%, more preferably 0.1% or less by weight of the stated ingredient. For example, the compositions of the invention that are substantially free of alcohol may contain less than 3% by weight of alcohol, preferably less than 1% by weight of alcohol, more preferably 0.1% or less alcohol.

A microorganism or microbe is an organism that is microscopic (too small to be seen by the human eye). Examples of microorganisms include bacteria, fungi, fungal spores, yeasts, moulds, mycobacteria, algae spores, bio-films, archaea and protists. Microorganisms are generally single-celled or unicellular organisms. However, as used herein, the term “microorganisms” also includes viruses.

By the term “anti-microbial” we mean a compound or composition that kills and/or inhibits the growth of microbes (microorganisms). As used herein, the term “anti-microbial” includes anti-bacterial, anti-biofilm, anti-fungal (fungal spores), anti-algal, anti-algal spores, anti-sporal, anti-viral, and/or anti-yeastal.

The compositions of the invention are anti-microbial. By this we mean that the compositions are anti-bacterial, anti-biofilm, anti-fungal (fungal spores), anti-algal, anti-algal spores, anti-sporal, anti-viral and/or anti-yeastal. As used herein, the terms anti-bacterial, anti-biofilm, anti-fungal (fungal spores), anti-algal, anti-algal spores, anti-sporal, anti-viral, and anti-yeastal agents are intended to refer to agents that inhibit the growth of the respective microorganisms but do not necessarily kill the microorganisms and agents that kill the respective microorganisms. Thus, for example, within the term anti-bacterial we include agents that inhibit the growth of bacteria but may not necessarily kill bacteria and bactericidal agents that do kill bacteria.

As the skilled person will appreciate, the word ending “cidal” as used in for example “bactericidal” and “fungicidal” is used to describe agents which kill the microorganism to which it refers. Thus, in these examples, bactericidal refers to an agent that kills bacteria and fungicidal refers to an agent that kills fungus. Examples of bactericides include myobactericides and tuberculocides.

The term “microbiocidal” is used to refer to compounds or compositions that kill microbes. As used herein, the term “microbiocidal” includes bactericidal, fungicidal, algacidal, sporicidal (eg; fungal sporicidal or algal sporicidal), viricidal, and/or yeasticidal.

The compositions of the invention may be microbiocidal. The compositions of the invention may be bactericidal, mycobactericidal, fungicidal, algicidal, sporicidal (eg; fungal sporicidal or algal sporicidal), viricidal, and/or yeasticidal. More preferably, the compositions of the invention are bactericidal, viricidal, fungicidal, fungal sporicidal and/or yeasticidal.

By the term “anti-viral” we mean a compound or composition that kills and/or inhibits the growth of viruses. The term “viricidal” is used to refer to compounds that kill viruses.

Some compositions of the invention are effective against a wide range of organisms, including Gram negative and Gram positive bacteria, mycobacteria, fungi, fungal spore, yeasts, viruses and/or some spore formers.

In the compositions of the invention, the active agent component (i) comprises
(A) a compound of formula (A)

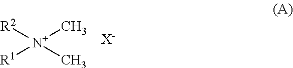

wherein R¹ and R² are independently C₈₋₁₂ alkyl group, such as C₈₋₁₀ alkyl or C₁₀₋₁₂ alkyl and X⁻ is a halide anion such as chloride, bromide, fluoride, iodide or sulphonate, saccharinate, carbonate or bicarbonate;
(B) a compound of formula (B)

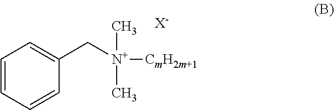

wherein m is from 8 to 18, and X⁻ is a halide anion such as chloride, bromide, fluoride, iodide or sulphonate, saccharinate, carbonate or bicarbonate; and
(C) a compound of formula (C)

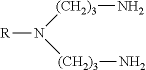

where R is an unsubstituted C₈ to C₁₈ alkyl group.

Commercially available blends of quaternary ammonium compounds having anti-microbial properties may be used as component (A) or component (B).

The quaternary ammonium anti-microbial agent(s) used in the present invention are typically water soluble at room temperature and pressure.

In the compounds of formula (A)

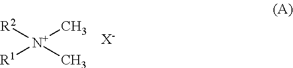

R¹ and R² may be each independently a straight chain, unsubstituted and uninterrupted C₈₋₁₂ alkyl group.

For example, R¹ and R² may be each independently a straight chain, unsubstituted and uninterrupted C₈₋₁₀ alkyl group or C₁₀₋₁₂ alkyl group. R¹ and R² may be each independently a straight chain, unsubstituted and uninterrupted C₈, C₉, C₁₀, C₁₁, or C₁₂ alkyl group.

X⁻ is a halide anion such as chloride, bromide, fluoride, iodide or sulphonate, saccharinate, carbonate or bicarbonate. In a preferred aspect, X is halide, more preferably a chloride anion.

In the compounds of formula (B)

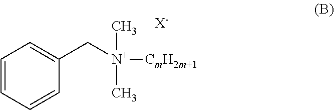

m is from 8 to 18. For example, m may be from 12 to 16 or may be from 12 to 14. m may be 8, 9, 10, 11, 12, 13, 14, 15, and/or 16.

The alkyl group present in the compounds of formula B may be a straight chain, unsubstituted and uninterrupted alkyl group.

X⁻ is a halide anion such as chloride, bromide, fluoride, iodide, sulphonate, saccharinate, carbonate or bicarbonate. In a preferred aspect, X⁻ is a halide, more preferably a chloride anion.

The compounds of formula (B) are generally called benzalkonium compounds. The benzalkonium chloride may be provided and/or used as a mixture of C₈₋₁₈ alkyl groups, particularly a mixture of straight chain, unsubstituted and uninterrupted alkyl groups n-C₈H₁₇ to n-C₁₈H₃₇, mainly n-C₁₂H₂₅ (dodecyl), n-C₁₄H₂₉ (tetradecyl), and n-C₁₆H₃₃ (hexadecyl).

The anion of each quaternary ammonium compound in the compositions of the invention may be the same or different. For example, the anion of a compound of formula (A) may be the same or different to the anion of a compound of formula (B). In one aspect, the anion for the compound of formula (A) and/or (B) is chloride.

Examples of quaternary ammonium compounds of formula (A) include di-n-decyldimethyl ammonium chloride (DDAC), octyl decyl dimethyl ammonium chloride and dioctyl dimethyl ammonium chloride.

Examples of commercially available compounds of formula (A) include Acticide DDQ 50 and Acticide DDQ 40 from Thor, Bardac 2250 and Bardac 2280 from Lonza and BTC101-E from Stepan.

Examples of quaternary ammonium compounds of formula (B) include alkyl (C12-16) dimethylbenzyl ammonium chloride (benzalkonium chloride, ADBAC), N,N-benzyldimethyloctylammonium chloride, N,N-benzyldimethyldecylammonium chloride, N-dodecyl-N-benzyl-N, N-dimethylammonium chloride, N-tetradecyl-N-benzyl-N,N-dimethylammonium chloride, N-hexadecyl-N,N-dimethyl-N-benzylammonium chloride, N,N-dimethyl N-benzyl N-octadecyl ammonium chloride and mixtures thereof.

Examples of commercially available compounds of formula (B) include Acticide BAC50 and Acticide BAC80 from Thor, Barquat mb-50 from Lonza and BTC50E from Stepan.

Commercially available compounds of formulas A, B and C often contain a mixture of compounds with different alkyl chain lengths. Examples of commercially available compounds of formula A, B and C that may be used in the present invention are shown below. It will be appreciated that other commercially available compounds may alternatively or additionally be used.

Didecyldimethylammonium chloride (DDAC): CAS number 7173-51-5 Didecyldimethylammonium chloride (DDAC (C8-10)): CAS number 68424-95-3

Alkyl (C12-16) dimethylbenzyl ammonium chloride (ADBAC (C12-C16)): CAS number 68424-85-1

Alkyl (C12-18) dimethylbenzyl ammonium chloride (ADBAC (C12-18)): CAS number 68391-01-5

Alkyl (C12-C14) dimethylbenzyl ammonium chloride (ADBAC (C12-C14)): CAS number 85409-22-9

It will be appreciated that a single CAS number often refers to more than one blend or mixture. A CAS classification for a commercial preparation typically covers blends comprising specified compounds in amounts within defined ranges. The compositions having the CAS numbers quoted above are only examples of compositions having a given CAS number that may be used in the present invention.

In the compositions of the invention, compound of formula (C) has the following formula:

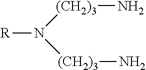

wherein R is an unsubstituted C₈ to C₁₈ alkyl group. Preferably R is from 10 to 14, for example 12. A preferred compound of formula (C) is N-(3-aminopropyl)-N-dodecylpropane-1,3-diamine (also known as dodecyl dipropylene triamine or (N,N-bis(3-aminopropyl)-dodecylamine, CAS no. 2372-82-9), commercially available from Lonza as Lonzabac 12, it is also known as Triameen YD12 (available from Nouryon).

In the active agent component, the amount of compounds (A), (B) and (C) will vary depending on a number of factors, such as the intended use of the composition and the particular compounds used.

In the composition of the invention (C) is present in an amount of from 5 to 95 wt % of the active agent component; and the ratio by weight of A:B is 1:9 to 9:1.

For example, (C) may be present in an amount from about 10 to about 90 wt % of the active agent component (i) and/or the ratio by weight of A:B is from 1:5 to 5:1. In a preferred aspect, (C) may be present in an amount from about 10 to about 90 wt % of the active agent component (i) and the ratio by weight of A: B is from 1:5 to 5:1.

(C) may be present in an amount of from 10 to 45 wt % of the active agent component; and/or the ratio by weight of A:B is 0.4:1 to 1:0.4. In a preferred aspect, (C) may be present in an amount from about 10 to about 45 wt % of the active agent component (i) and the ratio by weight of A:B is from 0.4:1 to 1:0.4.

(C) may be present in an amount of from 20 to 40 wt % of the active agent component; and/or the ratio by weight of A:B is 3:5 to 5:3. In a preferred aspect, (C) may be present in an amount from about 20 to about 40 wt % of the active agent component (i) and the ratio by weight of A:B is from 3:5 to 5:3.

(C) may be present in an amount of from 45 to 90 wt % of the active agent component; and/or the ratio by weight of A:B is 0.4:1 to 1:0.4. In a preferred aspect, (C) may be present in an amount from about 45 to about 90 wt % of the active agent component (i) and the ratio by weight of A:B is from 0.4:1 to 1:0.4.

(C) may be present in an amount of from 55 to 75 wt % of the active agent component; and/or the ratio by weight of A:B is 3:5 to 5:3. In a preferred aspect, (C) may be present in an amount from about 55 to about 75 wt % of the active agent component (i) and the ratio by weight of A:B is from 3:5 to 5:3.

Component (i) may consist essentially of or consist of a compound of formula (A), (B) and (C).

The compositions of the invention may additionally comprise any other suitable anti-microbial agent(s) (b), such as those described in the EPA (United States Environmental Protection Agency) Listing and Article 95 of the Biocidal Products Regulations (BPR Regulation (EU)528/2012).

Suitable anti-microbial agents (b) include anti-microbial agents that are not quaternary ammonium compounds. Preferably, these additional antimicrobial agent(s) are water soluble at room temperature and pressure.

Examples of suitable additional antimicrobial agents include but are not limited to polymeric biguanidines (e.g. polyhexamethylene biguanidine (PHMB)), non-polymeric biguanides (e.g. chlorhexidine), silver, octenidine HCl, amphoteric compounds, iodophores, phenolic compounds, amine anti-microbial agents and nitrogen based heterocyclic compounds, ortho phenyl phenol (OPP), and nitro bromopropanes (e.g. bronopol (INN), 2-bromo-2-nitropropane-1,3-diol), naturally derived biocidal compounds (e.g. honey and extracts of honey such as those comprising methyl glyoxal, flavenoids based antimicrobials, essential oils and organic acids e.g. lactic acid or citric acid).

In one aspect, the compositions of the invention are free of inorganic antimicrobial agents such as those comprising silver. In this aspect, the additional antimicrobial agent is an organic antimicrobial agent.

In one aspect, the compositions of the invention do not comprise PHMB or they may be free of polymeric biguanides or they may be free of non-polymeric biguanidines or free of biguanidines (eg free of polymeric and non-polymeric biguanides).

In one aspect of the invention the anti-microbial composition are substantially free of or does not comprise any isothiazole, isothiazlones and/or any nitrobromopropanes such as bronopol and/or any hypochlorites. In this aspect, the compositions of the invention are free of isothiazole, isothiazlones and/or any nitrobromopropanes such as bronopol and/or any hypochlorites. In other compositions of the invention, one or more of these compounds may be present as a preservative. The total amount of any such preservative would typically be no more that 0.1% by weight of the composition (for example no more that 0.1% by weight of the concentrated composition or no more that 0.1% by weight of the ready to use composition).

An example of a composition of the invention is a composition in which component (i) may comprise (A) di-n-decyldimethyl ammonium chloride (DDAC) as a compound of formula (A); and/or alkyl (C12-16) dimethylbenzyl ammonium chloride (benzalkonium chloride, ADBAC) as a compound of formula (B); and/or N-(3-aminopropyl)-N-dodecylpropane-1,3-diamine as a compound of formula (C).

Preferably, (A) is di-n-decyldimethyl ammonium chloride (DDAC); and (B) is alkyl (C12-16) dimethylbenzyl ammonium chloride (benzalkonium chloride, ADBAC); and (C) is N-(3-aminopropyl)-N-dodecylpropane-1,3-diamine.

The composition of the invention may be as defined in the paragraph above, with the concentrations and/or ratios as previously defined.

For example, the compositions of the invention may comprise (A) di-n-decyldimethyl ammonium chloride (DDAC) as a compound of formula (A); and/or alkyl (C12-16) dimethylbenzyl ammonium chloride (benzalkonium chloride, ADBAC) as a compound of formula (B); and/or N-(3-aminopropyl)-N-dodecylpropane-1,3-diamine as a compound of formula (C), wherein (C) may be present in an amount from about 10 to about 90 wt % of the active agent component (i) and/or the ratio by weight of A:B is from 1:5 to 5:1. In a preferred aspect, (C) may be present in an amount from about 10 to about 90 wt % of the active agent component (i) and the ratio by weight of A:B is from 1:5 to 5:1.

The composition of the invention may also comprise (A) di-n-decyldimethyl ammonium chloride (DDAC) as a compound of formula (A); and/or alkyl (C12-16) dimethylbenzyl ammonium chloride (benzalkonium chloride, ADBAC) as a compound of formula (B); and/or N-(3-aminopropyl)-N-dodecylpropane-1,3-diamine as a compound of formula (C), wherein (C) may be present in an amount of from 10 to 45 wt % of the active agent component; and/or the ratio by weight of A:B is 0.4:1 to 1:0.4. In a preferred aspect, (C) may be present in an amount from about 10 to about 45 wt % of the active agent component (i) and the ratio by weight of A:B is from 0.4:1 to 1:0.4.

The composition of the invention may also comprise (A) di-n-decyldimethyl ammonium chloride (DDAC) as a compound of formula (A); and/or alkyl (C12-16) dimethylbenzyl ammonium chloride (benzalkonium chloride, ADBAC) as a compound of formula (B); and/or N-(3-aminopropyl)-N-dodecylpropane-1,3-diamine as a compound of formula (C), wherein (C) may be present in an amount of from 20 to 40 wt % of the active agent component; and/or the ratio by weight of A:B is 3:5 to 5:3. In a preferred aspect, (C) may be present in an amount from about 20 to about 40 wt % of the active agent component (i) and the ratio by weight of A:B is from 3:5 to 5:3.

The composition of the invention may also comprise (A) di-n-decyldimethyl ammonium chloride (DDAC) as a compound of formula (A); and/or alkyl (C12-16) dimethylbenzyl ammonium chloride (benzalkonium chloride, ADBAC) as a compound of formula (B); and/or N-(3-aminopropyl)-N-dodecylpropane-1,3-diamine as a compound of formula (C), wherein (C) may be present in an amount of from 45 to 90 wt % of the active agent component; and/or the ratio by weight of A:B is 0.4:1 to 1:0.4. In a preferred aspect, (C) may be present in an amount from about 45 to about 90 wt % of the active agent component (i) and the ratio by weight of A:B is from 0.4:1 to 1:0.4.

The composition of the invention may also comprise (A) di-n-decyldimethyl ammonium chloride (DDAC) as a compound of formula (A); and/or alkyl (C12-16) dimethylbenzyl ammonium chloride (benzalkonium chloride, ADBAC) as a compound of formula (B); and/or N-(3-aminopropyl)-N-dodecylpropane-1,3-diamine as a compound of formula (C), wherein (C) may be present in an amount of from 55 to 75 wt % of the active agent component; and/or the ratio by weight of A:B is 3:5 to 5:3. In a preferred aspect, (C) may be present in an amount from about 55 to about 75 wt % of the active agent component (i) and the ratio by weight of A:B is from 3:5 to 5:3.

In an aspect of the compositions of the invention, it may be preferred that compound (C) is present in an amount of 20% or greater by weight of the composition. That is, it may be preferred that the compositions of the invention do not comprise less than 20% of compound (C) by weight of the composition.

The compositions of the invention are effective against a range of microorganisms, including viruses. However, depending on the specific nature of application of the product there may be more emphasis placed on its effectiveness against one or more organism. For example, for a product applied in a bathroom or damp environments there may be a bigger emphasis on fungus. Consequently, for certain specific uses other compositions and ratios of the biocides may be adjusted to be most beneficial.

For example, for a biocidal composition where there is more emphasis on anti-fungal efficacy and less so on anti-bacterial or anti-yeast efficacy, the optimal composition may move to higher ratios of N-(3-aminopropyl)-N-dodecylpropane-1,3-diamine relative to di-n-decyldimethyl ammonium chloride (DDAC) and alkyl dimethylbenzyl ammonium chloride (benzalkonium chloride, ADBAC) where the level of N-(3-aminopropyl)-N-dodecylpropane-1,3-diamine relative to di-n-decyldimethyl ammonium chloride (DDAC) and alkyl dimethylbenzyl ammonium chloride (benzalkonium chloride, ADBAC) is up to 55% or 75% of the total biocidal level, or even up to 90% or 95% of the total biocidal level.

Similarly when there is greater emphasis placed on anti-bacterial and/or anti-yeast efficacy as opposed to anti-fungal it may be beneficial to move to lower ratios of N-(3-aminopropyl)-N-dodecylpropane-1,3-diamine relative to di-n-decyldimethyl ammonium chloride (DDAC) and alkyl dimethylbenzyl ammonium chloride (benzalkonium chloride, ADBAC) where the level of N-(3-aminopropyl)-N-dodecylpropane-1,3-diamine relative to di-n-decyldimethyl ammonium chloride (DDAC) and alkyl dimethylbenzyl ammonium chloride (benzalkonium chloride, ADBAC) is down to 10% or even 5% of the total biocidal level.

Similarly if there is greater emphasis placed on anti-bacterial efficacy it may be beneficial to extend the ratio of di-n-decyldimethyl ammonium chloride (DDAC) to alkyl dimethylbenzyl ammonium chloride (benzalkonium chloride, ADBAC) to higher ratios of di-n-decyldimethyl ammonium chloride (DDAC) relative to alkyl dimethylbenzyl ammonium chloride (benzalkonium chloride, ADBAC), namely to include the range 3:5 to 1:0.4 respectively or depending on the degree of emphasis from 3:5 to 5:1 or from 3:5 to 9:1 respectively.

Similarly if there is greater emphasis placed on anti-yeast efficacy it may be beneficial to extend the ratio of di-n-decyldimethyl ammonium chloride (DDAC) to alkyl dimethylbenzyl ammonium chloride (benzalkonium chloride, ADBAC) to higher ratios of alkyl dimethylbenzyl ammonium chloride (benzalkonium chloride, ADBAC) relative to di-n-decyldimethyl ammonium chloride (DDAC), to include the range 0.4:1 to 5:3 respectively or depending on the degree of emphasis from 1:5 to 5:3 or from 1:9 to 5:3 respectively.

The compositions of the invention can be provided in concentrated form for dilution before use or in ready to use form. Unless otherwise stated, information about amounts (such as weight % or ppm) provided in this document relate to amounts at the point of use (for example the amount in a ready to use composition).

The total amount of the active agent component (i) in a composition of the invention at the point of use can be varied depending on the intended use.

For example, if the primary purpose of the use of the composition is anti-bacterial the amount of component (i) in the composition will typically be at least 300 ppm, such as at least 500 ppm or 300 to 1000 ppm or 400 to 800 ppm.

For example, if the primary purpose of the use of the composition is anti-yeastal the amount of component (i) in the composition will typically be at least 2000 ppm, such as at least 3000 ppm or 2000 to 10000 ppm or 2500 to 5000 ppm.

For example, if the primary purpose of the use of the composition is anti-fungal (fungal spores) the amount of component (i) in the composition will typically be at least 6000 ppm, such as at least 7000 ppm or 6000 to 1500 ppm or 7000 to 10000 ppm, such as about 8000 ppm.

For example, if the primary purpose of the use of the composition is anti-viral the amount of component (i) in the composition will typically be at least 300 ppm, such as at least 500 ppm or 300 to 5000 ppm or 400 to 1000 ppm.

It will be appreciated by the skilled reader that a composition containing component (i) in an amount higher than the ranges stated above for a particular type of micro-organism will still be effective against that type micro-organism. So, for example, a composition that contains component (i) in an amount to provide anti-yeastal properties will also be effective against bacteria and a composition that contains component (i) in an amount to provide anti-fungal (fungal spores) properties will also be effective against bacteria and yeasts.

There are significant advantages in terms of cost and environmental impact in the ability to dilute an anti-microbial composition so the actives are present in an amount needed to provide a required effect rather than using a large excess of actives.

Typically, the compositions of the invention do not comprise a polymer. For example, the compositions of the invention do not comprise hydrophilic polymers comprising at least two of the following types of monomer:
(1) a monomer having a permanent cationic charge or a monomer that is capable of forming a cationic charge upon protonation;
(2) an acidic monomer having an anionic charge or capable of forming an anionic charge; and
(3) a neutral monomer.

For example, the compositions of the invention do not comprise a polymer that comprises at least two of the following types of monomer:
(1) a N,N,N-trimethyl-3-((2-methyl-1-oxo-2-propenyl)amino)-1-propanaminium halide monomer or a diallyldimethylammonium halide monomer or a mixture thereof;
(2) an acidic monomer selected from acrylic acid and/or methylacrylic acid; and
(3) a neutral monomer selected from 2-(Dimethylamino)ethyl methacrylate (DMAEMA), N-vinyl pyrrolidone (NVP), N-vinylimidazole, acrylamide, or methacrylamide or a mixture thereof.

In particular, the compositions of the invention do not comprise polymers sold under the trade name Mirapol, for example as Mirapol Surf-S110, Mirapol Surf-S200 or Mirapol Surf-S500 available from Rhodia, Novecare.

The pH of the compositions of the invention can vary within wide limits. Typically, the pH of a composition of the invention will be similar to that of known compositions which are intended to be used for the same purpose or a similar purpose to a given composition of the invention. Formulations for use for purposes such as kitchen or bathroom cleaning may have either a low pH, such as a pH of 3 or below, for example about 2, or a high pH such as a pH of 10 or above, for example 11.

In one aspect, the compositions of the invention have a pH of 7 or more, for example 8 or more or 8.5 or more or pH 9.5 or more, such as from 8.5 to 11, for example from about pH 9.5 to about pH 10.5. For example, a concentrated composition may have a pH of from 9.5 to 11.5 and a ready to use composition may have a pH of from 8.5 to 10.5.

The compositions of the invention may comprise at least one surfactant. The compositions of the invention may comprise a non-ionic, cationic and/or amphoteric surfactant.

Suitable surfactants include non-ionic surfactants or a combination of surfactants including at least one non-ionic surfactant, for example at least one non-ionic surfactant optionally in combination with at least one cationic and/or amphoteric surfactant. The selection of surfactants will depend on the nature of and the intended purpose of the composition. Suitable surfactants for use in formulations intended for different purposes will be within the knowledge of the person of ordinary skill in the art. Likewise, the selection of suitable amounts of surfactant will be within the knowledge of the person of ordinary skill in the art.

Suitable non-ionic surfactants include, but are not limited to, amine oxides, alkyl polyglucosides, linear and branched 1° and 2° alcohol ethoxylates, nonyl phenol ethoxylates, and ethoxylated/propoxylated (EOPO) block polymers. In a preferred aspect, the surfactant is an alcohol ethoxylate, such as a linear and branched 1° and 2° alcohol ethoxylate.

Amine oxides suitable for use in the present invention are those with an alkyl chain length of C₈-C₁₆. Amine oxides are suitable for use in the present invention because they can provide better cleaning properties and can have less of a degradative effect on surfaces in particular plastic than some other surfactants.

Examples of suitable amine oxides compounds include, but are not limited to, C₈ amine oxide surfactants such as those sold under the trade name Macat AO-8 (Mason Chemical Company), C₁₀ amine oxides such as those sold under the trade names Euroxide D40 (EOC Group) and Mackamine C₁₀ (Rhodia Novecare), Longer chain glucosides such as C₁₂ amine oxides for example those sold under the trade names Euroxide LO/A (EOC Group), Ammonyx LO (Stepan Chemicals) and Surfac AO30 (Surfachem) and C₁₀-C₁₆ amine oxides such as those sold under the trade name Ammonyx C (Stepan Chemicals).

Alcohol alkoxylates suitable for use in the present invention include primary and secondary linear and branched alcohol ethoxylates, such as C₆-C₁₈ alcohol ethoxylates having 2 to 20 EO units such as 7 to 12 EO units. Preferred primary linear alcohol alkoxylates include C₉₋₁₁ alcohol ethoxylates having 5 to 12 EO units such as 7 to 12 EO units. These surfactants are available under the commercial name of the Neodol series (such as Neodol 91-8, Alcohols, C9-11, ethoxylated, 8EO, CAS number 68439-46-3 or Neodol 91-6, Alcohols, C9-11, ethoxylated, 6EO, also CAS number 68439-46-3, from Shell Chemical Company). Examples of alcohol ethoxylates with 20 EO units include SP BRIJ CS20 MBAL-PA(RB) (available from Croda), Alcohols, C16-18, ethoxylated, 20EO, CAS number 68439-49-6. Examples of secondary C12-15 alcohol ethoxylates, include those that have from about 3 to about 10 EO units, such those available in the Tergitol series (Dow), particularly those in the Tergitol 15-S series. Examples of suitable branched alcohol exthoxylates include C10 Guerbet alcohol with 3 to 14 EO units such as Lutensol XL and XP series (available from BASF). Alcohol ethoxylates having EO and PO mixtures are also suitable, these include linear and branched alcohol ethoxylates with PO-EO units such as ECOSURF™ EH Surfactants (DOW) and Plurafac D250 (BASF). Alkylphenol ethoxylate (APE) surfactants may also be used.

A further class of non-ionic surfactants that may be used is alkoxy block copolymers, in particular, compounds based on ethoxy/propoxyl block copolymers. Polymeric alkylene oxide block copolymers include non-ionic surfactants in which the major portion is made up of block polymeric C₂₋₄ alkylene oxides. Specific examples include surfactants commercially available under the Pluronic trade name and in particular the Pluronic F series, Pluronic L series, Pluronic P series and Pluronic R series.

A further class of suitable non-ionic surfactants are alkyl polyglucosides or glucosides. Suitable examples include Surface APG a capryl glucoside available from Surfachem and the Glucopon seriers available from BASF (Glucopon 425N/HH (C8-14), Glucopon 215 (C8-10) and Glucopon 600 (C12-14) alkyl polyglucoside).

In one aspect of the invention, the compositions of the invention may be free of alcohol alkoxylate.

The compositions of the invention may alternatively or additionally be free of alkyl polyglucoside. For example, compositions of the invention may be free of alkyl polyglucoside (APG).

Suitable amphoteric surfactants include but are not limited to C₆-C₂₀ alkylamphoacetates or amphodiacetates (such as cocoamphoacetates), C₁₀-C₁₈ alkyldimethyl betaines, C₁₀-C₁₈ alkyl amidopropyldimethyl betaines. Examples include but are not limited to coconut amphoteric surfactant cocoamidopropyl betaine (CAPB) (Surfac B4, CAS 61789-40-9), coco imidazoline betaine, oleo amido propyl betaine, and tall oil imidazoline.

Some compositions of the invention comprise an amphoteric surfactant. If a combination of an amphoteric surfactant and a non-ionic surfactant is used the weight ratio of the two types of surfactant can vary within wide limits, for example from 1% of amphoteric surfactant to 99% of non-ionic surfactant to 99% of amphoteric surfactant to 1% of non-ionic surfactant, based on the total weight of surfactant. The amphoteric surfactant and the non-ionic surfactant may, for example, be used in approximately equal amounts by weight.

The non-ionic, cationic or amphoteric surfactant may be present in the compositions of the invention in an amount of from about 0.01 to 1% by weight in the ready to use form or from about 0.5 to 10% by weight in the concentrated form:

Without wishing to be bound by theory, it has been found that the surfactant component can have several effects on the properties of the compositions of the invention. The presence of a non-ionic surfactant can improve the cleaning ability of the composition, that is improve the removal of soils and dirt. It has been found that the use of an alcohol alkoxylate in combination with an amine oxide can in some situations improve cleaning performance. Additionally, the inclusion of non-ionic surfactants can improve the stability of the compositions of the invention.

In one aspect, the composition is substantially free of or free of anionic surfactant. In another aspect, the compositions of the invention do not comprise an amphoteric surfactant.

The compositions of the invention comprise a polar solvent, component (ii). Suitable polar solvents include, but are not limited to, water, alcohols, glycol ethers and mixtures thereof.

Suitable alcohols include, but are not limited to, straight or branched chain C₁ to C₅ alcohols, such as methanol, ethanol, n-propanol, iso-propanol, mixtures of propanol isomers, n-butanol, sec-butanol, tert-butanol, iso-butanol, mixtures of butanol isomers, 2-methyl-1-butanol, n-pentanol, mixtures of pentanol isomers and amyl alcohol (mixture of isomers), and mixtures thereof.

Preferred polar solvents for use in the compositions of the invention include, but are not limited to, water, ethanol, n-propanol, isopropanol, ethylene glycol ethers, propylene glycol ethers, butyl diglycol (BDG) and dipropylene glycol methyl ether (Trade name Dowanol DPM) and mixtures thereof. In one aspect, the composition comprises water or a mixture of water and one or more alcohols selected from the alcohols described above. In such mixtures, water is preferably the major component. The polar solvent may consist essentially of water or be water.

If the compositions of the invention comprise an alcohol, the alcohol is typically present in an amount lower than the amount necessary for the alcohol to provide an antimicrobial effect.

In one aspect, the compositions of the invention may be substantially free of alcohol. For example, the compositions may contain 1% or less by weight alcohol. For example, the compositions may contain less than 1% or less than 0.5% by weight or 0.1% by weight or less of an alcohol such as isopropanol. As an example, compositions of the invention may comprise no isopropanol or may comprise no alcohol.

The composition may comprise water or a mixture of water and one or more alcohols selected from the alcohols described above. In such mixtures, water is preferably the major component.

In one aspect, the compositions of the invention are free of glycol ethers, for example they are free of propylene glycol n-butyl ether and/or free of a binary solvent combination of a glycol with a linear primary alcohol.

The compositions of the invention may comprise a chelating agent. Any suitable chelating agent may be used. Suitable chelating agent include but are not limited to EDTA (Ethylenediaminetetraacetic acid), Gluconate, GLDA (Glutamic acid diacetic acid)—Trade name Dissolvine GL, EDDS (Ethylenediamine-N,N′-disuccinic acid), DPTA (Diethylenetriaminepentaacetic acid), HEDTA (Hydroxyethylethylenediaminetriacetic acid), MGDA (Methyl glycine diacetic acid)—Trade name Trilon M, PDTA (1,3-propylenediaminetetraacetic acid), and EDG (Ethanoldiglycineic acid) and mixtures thereof. If the chelating agent contains a counter ion that counter ion is preferably metallic. Suitable metallic counter ions include but are not limited to Na, Ca, Fe, K, Zn, Mg and Mn.

Preferred chelating agents are GLDA (Dissolvine GL-38, L-glutamic acid N,N-diacetic acid, tetrasodium salt; GLDA-Na4, CAS number 51981-21-6) and EDTA.

The chelating agent may be present in an amount of from about 0.01 to 1.2% by weight in the ready to use form or from about 1 to 6% by weight in the concentrated form.

It will be appreciated that the compositions of the invention can comprise other ingredients commonly used in the art. The nature of any other ingredients used will depend on the nature and intended purpose of the composition. The person of ordinary skill in the art will know which additional ingredients are suitable for use in compositions for different applications.

Additional ingredients that may be used in the compositions of the invention include, but are not limited to, hydrophobic materials such as siloxanes, bicarbonate salts such as sodium bicarbonate, sodium chloride, potassium chloride, triethylamine, triethylenetetramine tetraethylethylenediamine, tetramethylenediamine, piperazines, histadines, imidazoles, morpholines, aminoalcohols such as 2-aminoethanol and 2-amino-2-methyl-1-propanol (AMP) and triethanolamine, phosphate buffers, citrate buffers and salts thereof and mixtures thereof.

pH modifiers may be used. Suitable pH modifiers include but are not limited to acids such as citric, sulfamic, hydrochloric, phosphoric, nitric, lactic, formic, acetic, salicylic, methanoic, glycolic or gluconic acids or other mineral or organic acids or bases such as sodium or potassium hydroxide and carbonates such as sodium carbonate, potassium carbonate, bicarbonates such as sodium bicarbonate, potassium bicarbonate, triethanolamine or monoethanolamine and mixtures thereof.

The compositions of the invention may alternatively or additionally contain salts such as the halides of alkali metals or alkaline earth metals such NaCl or KCl. In some situations, the use of salts can facilitate the formation of a stable composition.

Some compositions of the invention are free of or substantially free of acids or bases. For example, some compositions of the invention do not comprise a citrate or citric acid or lactic acid. Other compositions may contain an acid or base as is appropriate for their intended use. In one aspect of the invention the composition contains 0.5% by weight or less of formic acid or is free of formic acid.

In a particular aspect of the invention, the compositions of the invention do not comprise or are substantially free of a hydrophobic material, for example they do not comprise or are substantially free of siloxanes, silicones, polysiloxanes such as polydimethylsiloxanes.

The compositions of the invention may also contain other ingredients that are standard in the art such as colorants, fragrances, emollients, antioxidants, thickeners and corrosion inhibitors and mixtures thereof.

In a particular aspect of the invention, the compositions of the invention do not comprise or are substantially free of scale inhibitors. Scale inhibitors that preferably are not present in the compositions of the invention include amino carboxylates, amino phosphonates, polyfunctionally-substituted aromatic chelating agents.

The active agent component (i) may be present in the compositions of the invention in an amount of from about 0.01 to 12% by weight, such as 0.02 or 0.03 to 10% by weight or 0.05 to 8.0% by weight in the ready to use form or from about 2.0 to 50% by weight, such as 5.0 to 30% by weight or 10 to 20% by weight in the concentrated form.

The polar solvent component (ii) may be present in the compositions of the invention in an amount of from about 10 to about 99.999% by weight of the compositions. For example, in the concentrated form the polar solvent may be present in an amount of from about 10 to about 50% by weight of the composition, such as from about 15 to about 40% by weight of the composition or about 20 to about 30% by weight of the composition. In a ready to use composition of the invention, the polar solvent may be present in an amount of from about 50 to about 99.999% by weight of the composition, for example from about 80 to about 99.99% by weight of the composition, preferably from about 90 to about 99.9% by weight of the composition, more preferably from about 95 to about 99.8% by weight of the composition (e.g. from 97 to 99.7% or 97.5 to 99.6% by weight of the composition).

Without wishing to be bound by theory, the inventors have found that there are very significant advantages associated with the compositions of the invention.

It has been found that in use compositions of the invention have advantageous anti-microbial effects. For example, such compositions have an anti-microbial effect when initially applied to a surface (so called “wet kill”) and they can also have a residual effect in that they control or reduce viral particles/matter and/or reduce or prevent virus replication at the surface (so called “dry kill”). In particular, the compositions of the invention provide a residual anti-viral effect.

Some compositions of the invention may be resistant to washing with water and to wiping. This means that the compositions of the invention provide a residual anti-viral, effect even when the surface which has been treated is subsequently wiped and/or washed or rinsed with water.

An advantage of the invention is that it is possible to reduce or remove a broad range of microorganisms from a surface. The invention is therefore general in its control of microorganisms.

The invention also provides a composition that can be altered to target certain classes of micro-organism depending on the degree of dilution as discussed above.

The compositions of the invention can be used to control viruses or reduce viral particles/matter at the surface, for example by reducing or preventing virus replication.

The present invention also provides compositions as defined herein for use to reduce or remove microorganisms from the skin of a human or animal or the hair of a human or the fur of an animal.

The present invention also provides compositions as defined herein for use to reduce or control a virus or reduce virus replication on the skin of a human or animal or the hair of a human or the fur of an animal.

Typically, the compositions of the invention do not need to contain materials that are highly toxic to mammals. The anti-microbial agents used in the anti-microbial compositions are typically well known and widely understood and tested anti-microbial agents. The efficacy of the known anti-microbial agents is amplified in the formulations of the invention. Therefore, anti-microbial agents that have a low toxicity can be used in the anti-microbial compositions.

The anti-microbial compositions of the invention do not contain materials that produce highly persistent residues or rinsates or products that contain heavy metals and their salts. Thus, there is a greatly reduced risk of long-term hazards.

The anti-microbial compositions of the invention do not interfere with the biochemical reproductive pathways of the micro-organisms they control. The risk of resistance build up and the development of resistant strains is, therefore, low.

The anti-microbial compositions of the invention can have a dual effect in that not only do they provide an anti-microbial effect in use but they can also have a preservative effect on the composition. This means that it is typically not necessary to include additional preservatives in the formulations of the invention.

The compositions of the invention do not typically give surfaces to which they are applied a greasy feel.

There is also provided the use of a composition of the invention reduce the number of microbes on or at a surface.

Further, there is provided the use of a composition of the invention to reduce the number of microbes on or at a surface and to remove soil from that surface.

The present inventors have surprisingly found that by using the combinations described herein stable compositions that provide residual anti-viral, performance can be obtained. The present invention therefore provides use of a composition of the invention to reduce and/or control viruses on or at a surface.

Also provided is a method for reducing the number of microbes on or at a surface, which method comprises applying a composition of the invention a surface.

Further, the present invention provides a method for reducing the number of microbes on or at a surface and removing soil from that surface, which method comprises applying a composition of the invention to a surface.

Surfaces that may be treated using a composition of the invention are described above and include hard surfaces such as non-porous or porous hard surfaces and porous surfaces such as soft surfaces.

The method may be used to reduce and/or control viruses on or at a surface.

The present invention provides a method for providing a residual anti-viral benefit to a surface as defined above such as a hard or porous surface, which method comprises applying a composition as defined herein to that surface. The composition may be applied to the surface by spraying the composition on the surface or wiping the composition onto the surface.

In one method of the invention, it is not necessary for the method to include any steps in addition to simply applying the composition to the surface. Thus, a method that consists essentially of or consists of applying the composition to the surface is provided.

It will be appreciated that in order to also provide cleaning it may also be necessary to wipe or scrub the surface. Thus, the invention also provides a method in which the surface is wiped or scrubbed when the composition of the invention is applied to the surface.

An anti-microbial wipe comprising a substrate and an anti-microbial composition of the invention is also provided.

The compositions of the invention can be used in the form in which they are provided or can be diluted with water before use. Thus, the invention also provides a method, such as a method as defined above, in which a composition of the invention is diluted before use.

The anti-microbial compositions of the invention can typically degrade when submersed in water, to provide a rinsate/leachate of low toxicity and which has a short residence time in the environment.

In one particular aspect, the invention provides a composition comprising an active agent component comprising compounds A, B and C as defined above and a non-ionic surfactant and a chelating agent. Preferably, (A) is di-n-decyldimethyl ammonium chloride (DDAC); and (B) is alkyl (C12-16) dimethylbenzyl ammonium chloride (benzalkonium chloride, ADBAC); and (C) is N-(3-aminopropyl)-N-dodecylpropane-1,3-diamine and the surfactant is a primary and secondary linear and branched alcohol ethoxylates, such as C₆-C₁₈ alcohol ethoxylates having 2 to 20 EO units and the chelating agent is GLDA.

The anti-microbial compositions of the invention are typically made by a process as described below.
1. The polar solvent (component (ii)) is weighed into a suitably sized vessel;
2. The required amount of the biocide(s) (component (A, B, C)), is then added to the vessel;
3. Optionally a surfactant may be added to the vessel
4. Optionally a chelating agent may be added to the vessel
5. Optionally the pH of the composition may be checked and adjusted as necessary;
6. The vessel is then made up to 100% by weight by the addition of further polar solvent as required.

The present invention provides compositions obtainable by the process set out above.

### BRIEF DESCRIPTION OF THE FIGURES

FIG. 1: Shows the triangular coordinate of a composition comprising di-n-decyldimethyl ammonium chloride (DDAC), alkyl dimethylbenzyl ammonium chloride (benzalkonium chloride, ADBAC) and N-(3-aminopropyl)-N-dodecylpropane-1,3-diamine (Triameen). The point denoted by “A” represents 20% by weight DDAC, 50% by weight Triameen and 30% by weight ADBAC.

FIG. 2: Shows the ratio of di-n-decyldimethyl ammonium chloride (DDAC), alkyl dimethylbenzyl ammonium chloride (benzalkonium chloride, ADBAC) and N-(3-aminopropyl)-N-dodecylpropane-1,3-diamine through triangular coordinates, present in compositions 14.1 to 14.17. Full compositions for 14.1 to 14.17 are provided in Table 1.

FIG. 3: Shows Log reduction (LogR) values (as shown in Table 2) for anti-microbial efficacy against pseudomonas overlaid on triangular coordinates for Compositions 14.1 to 14.17 diluted at 0.6%.

FIG. 4: Shows Log reduction (LogR) (as shown in Table 3) for anti-microbial efficacy against Candida albicans overlaid on triangular coordinates for Compositions 15.1 to 15.17 diluted at 3.0%.

FIG. 5: Shows Log reduction (LogR) (as shown in Table 4) for anti-microbial efficacy against aspergillus brasilliensis overlaid on triangular coordinates for Compositions 14.1 to 14.17 diluted at 8.0%.

The invention will now be illustrated by the following non-limiting Examples.

### Example 1—Preparation of Compositions of the Invention and Comparative Compounds

The compositions as described in Table 1 were prepared using the method of described below. The commercially available compounds used in the compositions are listed below.

Acticide DDQ 40 supplied by Thor (DDAC): contains 40% di-n-decyldimethyl ammonium chloride (DDAC). This is an example of component A of the invention.

Acticide BAC 50M supplied by Thor (ADBAC): contains 50% alkyl (C12-16) dimethylbenzyl ammonium chloride (benzalkonium chloride, ADBAC) is an example of component B of the invention.

Triameen Y12D-30, supplied by Nouryon: contains 30% N-(3-aminopropyl)-N-dodecylpropane-1,3-diamine is an example is an example of component C of the invention.

Neodol 91-8 supplied by Shell is Alcohols, C9-11, ethoxylated, 8EO

SP BRIJ CS20 MBAL-PA(RB) supplied by Croda is Alcohols, C16-18, ethoxylated, 20EO

Dissolvine GL-38 supplied by Nouryon: contains 30% L-glutamic acid N,N-diacetic acid, tetrasodium salt; GLDA-Na4

Purac FCC 80 supplied by Corbion: contains 80% L-lactic acid

Sodium carbonate supplied by Vickers Laboratories

Sodium bicarbonate supplied by Scientific Laboratory Supplies (SLS)

The polar solvent (component (ii)) was placed in a suitably sized vessel (typically of 150 millilitre capacity). A small amount of the polar solvent was retained to allow for final weight adjustment. The required amount of the biocide(s) (component (A, B, C)), was then added to the vessel, with stirring for 5 minutes after addition of each biocide before the next biocide is added. The required amount of the surfactant was then added and the vessel stirred for a further 45 minutes. The required amount of the chelant was then added and the vessel stirred for a further 5 minutes. The pH was checked and was adjusted using lactic acid solution, sodium carbonate and sodium bicarbonate as required. The solution was then made up to 100% by weight by adding the retained polar solvent and stirred for a final 10 minutes.

The Comparative Examples were prepared following the same method but by varying the levels of components A, B or C or by substituting the surfactant component.

The compositions produced by this method were “concentrated” compositions which were diluted prior to use as specified in Tables 2, 3 and 4 below.

### Example 2—Evaluation of Antimicrobial Activity

The evaluation of antimicrobial activity was assessed on hard non porous surfaces (stainless steel discs) based on the European Standard EN 13697:2015+A1:2019: Chemical disinfectants and antiseptics, Quantitative non-porous surface test for the evaluation of bactericidal and/or fungicidal activity of chemical disinfectants used in food, industrial, domestic and institutional areas. Test method and requirements without mechanical action (phase 2, step 2).

The microoroganisms tested were Pseudomonas aeruginosa NCTC 13359 (bacteria), Candida albicans NCPF 3179 (yeast), and spores of Aspergillus brasiliensis ATCC 16404 (fungi).

Test microorganisms were cultured and fungal spores prepared as described in EN 13697:2015+A1:2019. A modification was made to the EN 13697 method in order to test against the microorganisms and fungal spores in the presence of bovine albumin and sheep erythrocytes, which is representative of testing under ‘dirty’ conditions in the medical area. For preparation of sheep erythrocytes and bovine albumin, the procedures used in EN 16777: 2018 Chemical disinfectants and antiseptics—Quantitative non-porous surface test without mechanical action for the evaluation of virucidal activity of chemical disinfectants used in the medical area—Test method and requirements (phase 2/step 2) were followed. The final concentration of sheep erythrocytes and albumin in the test procedure was 3 ml/l and 3 g/l respectively.

Nine parts of the bacterial, yeast, or fungal spore suspension were mixed with one part of the albumin/erythrocytes (soil) suspension prior to testing. Fifty microlitres of this microbial/soil suspension was then added to a stainless-steel disc and allowed to dry in a controlled manner. Normally at 37° C. in a laboratory oven (P. aeruginosa and C. albicans). The Aspergillus brasiliensis/soil suspension were dried onto stainless steel discs in a containment level 2 cabinet at ambient temperature. Once dried, 100 microlitres of test formulation (or sterile deionised water for control) was added to the stainless-steel discs at ambient temperature (˜20° C.).

Test formulations were prepared in standardised hard water (see EN 13697 for preparation of hard water) to their required % (v/v) test dilution. Following the required contact time (the time that the formulation or water control is in contact with the microbial/soil suspension), the stainless steel discs were transferred into a sterile tube containing 5 g sterile glass beads (diameter: ≤5 mm) and 10 ml of neutralisation solution (per litre deionised water) lecithin, 11.68 g; polysorbate 80, 100 ml; sodium dodecyl sulphate, 10 g; sodium thiosulphate, 5 g; tryptone, 1 g; sodium chloride, 8.5 g. Surviving microorganisms were recovered from the discs by vortex mixing the neutralisation solution containing the disc for 60 seconds. The vortexed mixture was then further diluted accordingly in neutralisation solution and pipetted as 1 ml samples into Petri dishes which were then overlaid with tryptone soya agar (TSA) for bacteria, and with malt extract agar (MEA) for yeast and fungi. TSA plates were incubated at 37° C. for 24 hours, and MEA plates were incubated at 30° C. for 48 hours.

The resulting colonies were counted on the test and water control plates. From these values, the logarithmic reduction (LogR) achieved from the formulations were calculated by comparison with the water-treated control. The higher the logR achieved, the greater the efficacy of the formulation was under those test conditions. A logR value preceded by >means that total detectable reduction of challenge microorganism was reported in the test. The logR was likely to be higher than that reported, however cannot be specifically known due to limits of detection within the test. A value of TNTC means that microbial colonies were too numerous to count, therefore indicating a lack of antimicrobial activity in that test.

The results are shown below in Tables 1 to 4.

TABLE 1

| | | % by weight of each component in the Composition | | | | | |
|---|---|---|---|---|---|---|---|
| | di-n-decyldimethyl | alkyl | | tetrasodium | | | |
| | ammonium | dimethylbenzyl | N-(3-aminopropyl)- | glutamate | | | |
| | chloride | ammonium | N-dodecylpropane- | diacetate | Brij | Neodol | Sodium |
| Composition | (DDAC) | chloride | 1,3-diamine | (GLDA) | CS20 | 91-8 | carbonate |
| 14.1 | 3.89 | 0.00 | 1.11 | 4.00 | 1.00 | 0.00 | 0.3-0.6 |
| 14.2 | 2.78 | 1.11 | 1.11 | 4.00 | 1.00 | 0.00 | 0.3-0.6 |
| 14.3 | 1.94 | 1.94 | 1.11 | 4.00 | 1.00 | 0.00 | 0.3-0.6 |
| 14.4 | 1.11 | 2.78 | 1.11 | 4.00 | 1.00 | 0.00 | 0.3-0.6 |
| 14.5 | 0.00 | 3.89 | 1.11 | 4.00 | 1.00 | 0.00 | 0.3-0.6 |
| 14.6 | 1.67 | 0.00 | 3.33 | 4.00 | 1.00 | 0.00 | 0.3-0.6 |
| 14.7 | 0.83 | 0.83 | 3.33 | 4.00 | 1.00 | 0.00 | 0.3-0.6 |
| 14.8 | 0.00 | 1.67 | 3.33 | 4.00 | 1.00 | 0.00 | 0.3-0.6 |
| 14.9 | 2.78 | 0.00 | 2.22 | 4.00 | 1.00 | 0.00 | 0.3-0.6 |
| 14.10 | 2.22 | 0.56 | 2.22 | 4.00 | 1.00 | 0.00 | 0.3-0.6 |
| 14.11 | 1.39 | 1.39 | 2.22 | 4.00 | 1.00 | 0.00 | 0.3-0.6 |
| 14.12 | 0.56 | 2.22 | 2.22 | 4.00 | 1.00 | 0.00 | 0.3-0.6 |
| 14.13 | 0.00 | 2.78 | 2.22 | 4.00 | 1.00 | 0.00 | 0.3-0.6 |
| 14.14 | 0.00 | 0.00 | 5.00 | 4.00 | 1.00 | 0.00 | 0.3-0.6 |
| 14.15 | 0.00 | 5.00 | 0.00 | 4.00 | 1.00 | 0.00 | 0.8-1.5 |
| 14.16 | 5.00 | 0.00 | 0.00 | 4.00 | 1.00 | 0.00 | 0.8-1.5 |
| 14.17 | 2.50 | 2.50 | 0.00 | 4.00 | 1.00 | 0.00 | 0.8-1.5 |
| 15.1 | 3.89 | 0.00 | 1.11 | 4.00 | 0.00 | 1.00 | 0.3-0.6 |
| 15.2 | 2.78 | 1.11 | 1.11 | 4.00 | 0.00 | 1.00 | 0.3-0.6 |
| 15.3 | 1.94 | 1.94 | 1.11 | 4.00 | 0.00 | 1.00 | 0.3-0.6 |
| 15.4 | 1.11 | 2.78 | 1.11 | 4.00 | 0.00 | 1.00 | 0.3-0.6 |
| 15.5 | 0.00 | 3.89 | 1.11 | 4.00 | 0.00 | 1.00 | 0.3-0.6 |
| 15.6 | 1.67 | 0.00 | 3.33 | 4.00 | 0.00 | 1.00 | 0.3-0.6 |
| 15.7 | 0.83 | 0.83 | 3.33 | 4.00 | 0.00 | 1.00 | 0.3-0.6 |
| 15.8 | 0.00 | 1.67 | 3.33 | 4.00 | 0.00 | 1.00 | 0.3-0.6 |
| 15.9 | 2.78 | 0.00 | 2.22 | 4.00 | 0.00 | 1.00 | 0.6-0.8 |
| 15.10 | 2.22 | 0.56 | 2.22 | 4.00 | 0.00 | 1.00 | 0.6-0.8 |
| 15.11 | 1.39 | 1.39 | 2.22 | 4.00 | 0.00 | 1.00 | 0.6-0.8 |
| 15.12 | 0.56 | 2.22 | 2.22 | 4.00 | 0.00 | 1.00 | 0.6-0.8 |
| 15.13 | 0.00 | 2.78 | 2.22 | 4.00 | 0.00 | 1.00 | 0.6-0.8 |
| 15.14 | 0.00 | 0.00 | 5.00 | 4.00 | 0.00 | 1.00 | 0.8-1.5 |
| 15.15 | 0.00 | 5.00 | 0.00 | 4.00 | 0.00 | 1.00 | 0.8-1.5 |
| 15.16 | 5.00 | 0.00 | 0.00 | 4.00 | 0.00 | 1.00 | 0.8-1.5 |
| 15.17 | 2.50 | 2.50 | 0.00 | 4.00 | 0.00 | 1.00 | 0.8-1.5 |

| | % by weight of each component | | | PH of | PH of | PH of |
|---|---|---|---|---|---|---|
| | in the Composition | | pH | composition | composition | composition |
| | Sodium | Lactic | of | diluted | diluted | diluted |
| Composition | bicarbonate | acid | composition | to 0.6% | to 3.0% | to 8.0% |
| 14.1 | 0.1-0.2 | 0.2-0.4 | 9.7-10.4 | 8.4-9.1 | 8.9-9.7 | 9.5-10.3 |
| 14.2 | 0.1-0.2 | 0.2-0.4 | 9.7-10.4 | 8.4-9.1 | 8.9-9.7 | 9.5-10.3 |
| 14.3 | 0.1-0.2 | 0.2-0.4 | 9.7-10.4 | 8.4-9.1 | 8.9-9.7 | 9.5-10.3 |
| 14.4 | 0.1-0.2 | 0.2-0.4 | 9.7-10.4 | 8.4-9.1 | 8.9-9.7 | 9.5-10.3 |
| 14.5 | 0.1-0.2 | 0.2-0.4 | 9.7-10.4 | 8.4-9.1 | 8.9-9.7 | 9.5-10.3 |
| 14.6 | 0.1-0.2 | 1.1-1.4 | 9.7-10.4 | 8.4-9.1 | 8.9-9.7 | 9.5-10.3 |
| 14.7 | 0.1-0.2 | 1.1-1.4 | 9.7-10.4 | 8.4-9.1 | 8.9-9.7 | 9.5-10.3 |
| 14.8 | 0.1-0.2 | 1.1-1.4 | 9.7-10.4 | 8.4-9.1 | 8.9-9.7 | 9.5-10.3 |
| 14.9 | 0.1-0.2 | 1.1-1.4 | 9.7-10.4 | 8.4-9.1 | 8.9-9.7 | 9.5-10.3 |
| 14.10 | 0.1-0.2 | 1.1-1.4 | 9.7-10.4 | 8.4-9.1 | 8.9-9.7 | 9.5-10.3 |
| 14.11 | 0.1-0.2 | 1.1-1.4 | 9.7-10.4 | 8.4-9.1 | 8.9-9.7 | 9.5-10.3 |
| 14.12 | 0.1-0.2 | 1.1-1.4 | 9.7-10.4 | 8.4-9.1 | 8.9-9.7 | 9.5-10.3 |
| 14.13 | 0.1-0.2 | 1.1-1.4 | 9.7-10.4 | 8.4-9.1 | 8.9-9.7 | 9.5-10.3 |
| 14.14 | 0.1-0.2 | 2.0-2.2 | 9.7-10.4 | 8.4-9.1 | 8.9-9.7 | 9.5-10.3 |
| 14.15 | 0.2-0.4 | 0.2-0.4 | 9.7-10.4 | 8.4-9.1 | 8.9-9.7 | 9.5-10.3 |
| 14.16 | 0.2-0.4 | 0.2-0.4 | 9.7-10.4 | 8.4-9.1 | 8.9-9.7 | 9.5-10.3 |
| 14.17 | 0.2-0.4 | 0.2-0.4 | 9.7-10.4 | 8.4-9.1 | 8.9-9.7 | 9.5-10.3 |
| 15.1 | 0.1-0.2 | 0.2-0.4 | 9.7-10.4 | 8.4-9.1 | 8.9-9.7 | 9.5-10.3 |
| 15.2 | 0.1-0.2 | 0.2-0.4 | 9.7-10.4 | 8.4-9.1 | 8.9-9.7 | 9.5-10.3 |
| 15.3 | 0.1-0.2 | 0.2-0.4 | 9.7-10.4 | 8.4-9.1 | 8.9-9.7 | 9.5-10.3 |
| 15.4 | 0.1-0.2 | 0.2-0.4 | 9.7-10.4 | 8.4-9.1 | 8.9-9.7 | 9.5-10.3 |
| 15.5 | 0.1-0.2 | 0.2-0.4 | 9.7-10.4 | 8.4-9.1 | 8.9-9.7 | 9.5-10.3 |
| 15.6 | 0.1-0.2 | 1.1-1.4 | 9.7-10.4 | 8.4-9.1 | 8.9-9.7 | 9.5-10.3 |
| 15.7 | 0.1-0.2 | 1.1-1.4 | 9.7-10.4 | 8.4-9.1 | 8.9-9.7 | 9.5-10.3 |
| 15.8 | 0.1-0.2 | 1.1-1.4 | 9.7-10.4 | 8.4-9.1 | 8.9-9.7 | 9.5-10.3 |
| 15.9 | 0.1-0.2 | 1.1-1.4 | 9.7-10.4 | 8.4-9.1 | 8.9-9.7 | 9.5-10.3 |
| 15.10 | 0.1-0.2 | 1.1-1.4 | 9.7-10.4 | 8.4-9.1 | 8.9-9.7 | 9.5-10.3 |
| 15.11 | 0.1-0.2 | 1.1-1.4 | 9.7-10.4 | 8.4-9.1 | 8.9-9.7 | 9.5-10.3 |
| 15.12 | 0.1-0.2 | 1.1-1.4 | 9.7-10.4 | 8.4-9.1 | 8.9-9.7 | 9.5-10.3 |
| 15.13 | 0.1-0.2 | 1.1-1.4 | 9.7-10.4 | 8.4-9.1 | 8.9-9.7 | 9.5-10.3 |
| 15.14 | 0.1-0.2 | 2.0-2.2 | 9.7-10.4 | 8.4-9.1 | 8.9-9.7 | 9.5-10.3 |
| 15.15 | 0.2-0.4 | 0.3-0.6 | 9.7-10.4 | 8.4-9.1 | 8.9-9.7 | 9.5-10.3 |
| 15.16 | 0.2-0.4 | 0.3-0.6 | 9.7-10.4 | 8.4-9.1 | 8.9-9.7 | 9.5-10.3 |
| 15.17 | 0.2-0.4 | 0.3-0.6 | 9.7-10.4 | 8.4-9.1 | 8.9-9.7 | 9.5-10.3 |

The anti-microbial efficacy is expressed in terms of the log reduction (Log_{R}) achieved against the relevant organism and dilution of the composition. These results are shown in Table 2, 3 and 4 below for bacteria (Pseudomonas aeruginosa), yeast (Candida albicans) and fungus (Aspergillus brasiliensis) respectively.

TABLE 2

| | Dilution of | | Organism | Test Result |
|---|---|---|---|---|
| Composition | Composition | Test Number | tested | (Log_{R}) |
| 14.1 | 0.6% | T1.PA.14.1 | pseudomonas | 3.48 |
| 14.2 | | T1.PA.14.2 | aeruginosa | >6.17 |
| 14.3 | | T1.PA.14.3 | | >6.17 |
| 14.4 | | T1.PA.14.4 | | 2.84 |
| 14.5 | | T1.PA.14.5 | | 3.10 |
| 14.6 | | T1.PA.14.6 | | >6.17 |
| 14.7 | | T1.PA.14.7 | | >6.17 |
| 14.8 | | T1.PA.14.8 | | >6.17 |
| 14.9 | | T1.PA.14.9 | | >6.17 |
| 14.10 | | T1.PA.14.10 | | >6.17 |
| 14.11 | | T1.PA.14.11 | | >6.17 |
| 14.12 | | T1.PA.14.12 | | >6.17 |
| 14.13 | | T1.PA.14.13 | | >6.17 |
| 14.14 | | T1.PA.14.14 | | >6.17 |
| 14.15 | | T1.PA.14.15 | | 2.48 |
| 14.16 | | T1.PA.14.16 | | 2.52 |
| 14.17 | | T1.PA.14.17 | | 3.34 |

TABLE 3

| | Dilution of | | Organism | Test Result |
|---|---|---|---|---|
| Composition | Composition | Test Number | tested | (Log_{R}) |
| 15.1 | 3.0% | T1.CA.15.1 | Candida | 3.03 |
| 15.2 | | T1.CA.15.2 | albicans | 3.33 |
| 15.3 | | T1.CA.15.3 | | >6.15 |
| 15.4 | | T1.CA.15.4 | | >6.15 |
| 15.5 | | T1.CA.15.5 | | 2.18 |
| 15.6 | | T1.CA.15.6 | | >6.15 |
| 15.7 | | T1.CA.15.7 | | 6.15 |
| 15.8 | | T1.CA.15.8 | | 3.80 |
| 15.9 | | T1.CA.15.9 | | 3.36 |
| 15.10 | | T1.CA.15.10 | | 6.15 |
| 15.11 | | T1.CA.15.11 | | 6.15 |
| 15.12 | | T1.CA.15.12 | | >6.15 |
| 15.13 | | T1.CA.15.13 | | 6.15 |
| 15.14 | | T1.CA.15.14 | | 6.15 |
| 15.15 | | T1.CA.15.15 | | 2.44 |
| 15.16 | | T1.CA.15.16 | | TNTC |
| 15.17 | | T1.CA.15.17 | | 2.05 |

TABLE 4

| | Dilution of | | Organism | Test Result |
|---|---|---|---|---|
| Composition | Composition | Test Number | tested | (Log_{R}) |
| 14.1 | 8.0% | T1.AB.14.1 | aspergillus | 3.10 |
| 14.2 | | T1.AB.14.2 | brasilliensis | 2.61 |
| 14.3 | | T1.AB.14.3 | | 2.58 |
| 14.4 | | T1.AB.14.4 | | 2.53 |
| 14.5 | | T1.AB.14.5 | | 2.23 |
| 14.6 | | T1.AB.14.6 | | 2.36 |
| 14.7 | | T1.AB.14.7 | | 1.96 |
| 14.8 | | T1.AB.14.8 | | 2.04 |
| 14.9 | | T1.AB.14.9 | | 2.23 |
| 14.10 | | T1.AB.14.10 | | 2.18 |
| 14.11 | | T1.AB.14.11 | | 2.11 |
| 14.12 | | T1.AB.14.12 | | 2.08 |
| 14.13 | | T1.AB.14.13 | | 2.07 |
| 14.14 | | T1.AB.14.14 | | 1.84 |
| 14.15 | | T1.AB.14.15 | | 3.18 |
| 14.16 | | T1.AB.14.16 | | 3.31 |
| 14.17 | | T1.AB.14.17 | | 3.35 |

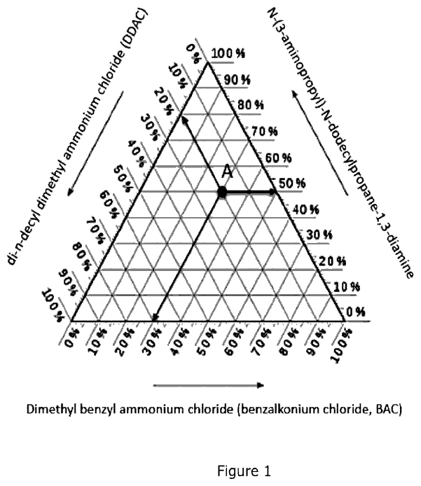

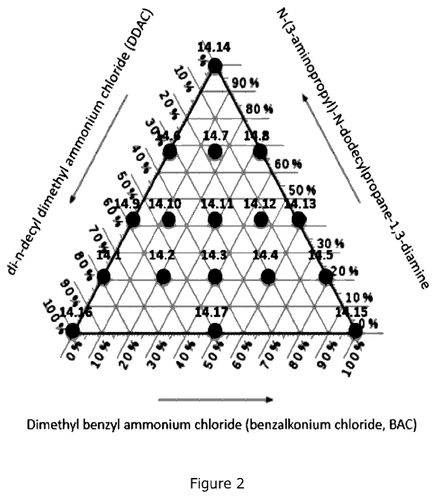

To understand better the trends in anti-microbial efficacy as the composition of the biocidal actives are varied, the inventors overlaid the results obtained in Tables 2, 3 and 4 onto a triangular compositional plots as shown in FIGS. 1 to 5.

As illustrated in the aforementioned data, the optimal compositions to maximise efficacy against bacteria, yeast and fungus are when the level of N-(3-aminopropyl)-N-dodecylpropane-1,3-diamine is between 20 and 40% of the total biocidal level, and when the ratio of di-n-decyldimethyl ammonium chloride (DDAC) to alkyl dimethylbenzyl ammonium chloride (benzalkonium chloride, ADBAC) is between 3:5 and 5:3 respectively.

The aforementioned ratios were found to be the optimal composition to be effective against all organisms, namely bacteria, yeast and fungus.

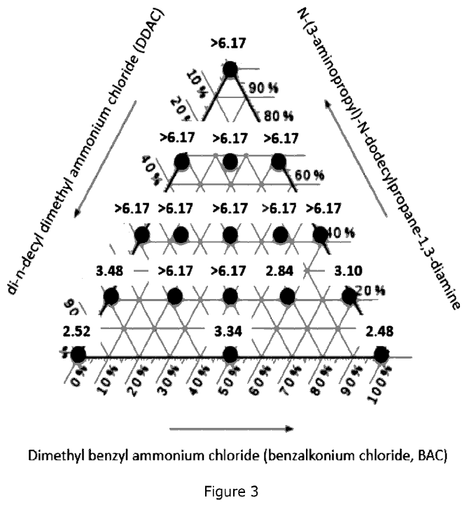

FIG. 3 shows the following trends for anti-microbial efficacy against bacteria. As the proportion of N-(3-aminopropyl)-N-dodecylpropane-1,3-diamine is increased relative to di-n-decyldimethyl ammonium chloride (DDAC) and alkyl dimethylbenzyl ammonium chloride (benzalkonium chloride, ADBAC) the anti-bacterial efficacy as represented by the LogR value increases and reaches a maximum value when the proportion of N-(3-aminopropyl)-N-dodecylpropane-1,3-diamine relative to di-n-decyldimethyl ammonium chloride (DDAC) and alkyl dimethylbenzyl ammonium chloride (benzalkonium chloride, BAC) is between 20% and 40%.

For example, compare Test Results (LogR values) from Tests T1.PA. 14.17, T1.PA. 14.3, T1. PA. 14.11, T1.PA. 14.7, T1. PA. 14.14 shown in Table 2.

When the proportion of N-(3-aminopropyl)-N-dodecylpropane-1,3-diamine relative to di-n-decyldimethyl ammonium chloride (DDAC) and alkyl dimethylbenzyl ammonium chloride (benzalkonium chloride, ADBAC) is below 40% the anti-bacterial efficacy as represented by the LogR values is improved by the presence of both di-n-decyldimethyl ammonium chloride (DDAC) and alkyl dimethylbenzyl ammonium chloride (benzalkonium chloride, ADBAC) as opposed to either alone and in particular when the ratio of di-n-decyldimethyl ammonium chloride (DDAC) to alkyl dimethylbenzyl ammonium chloride (benzalkonium chloride, ADBAC) is between 5:3 and 1:1 respectively.

For example, compare Test Results (LogR values) from Tests T1.PA.14.1, T1.PA.14.2, T1. PA. 14.3, T1.PA. 14.4, T1. PA. 14.5 shown in Table 2.

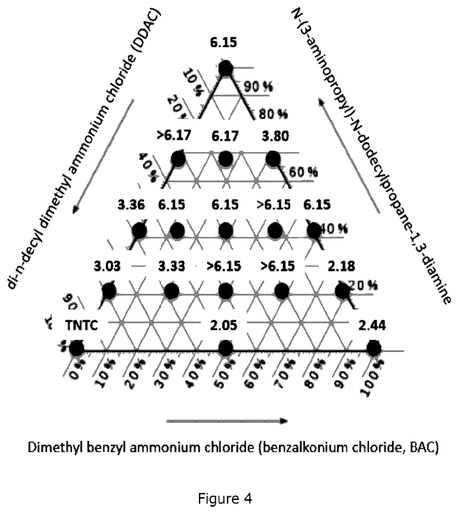

FIG. 4 shows the following trends for anti-microbial efficacy against yeast.

As the proportion of N-(3-aminopropyl)-N-dodecylpropane-1,3-diamine is increased relative to di-n-decyldimethyl ammonium chloride (DDAC) and alkyl dimethylbenzyl ammonium chloride (benzalkonium chloride, ADBAC) the anti-yeast efficacy as represented by the LogR value increases and reaches a maximum value when the proportion of N-(3-aminopropyl)-N-dodecylpropane-1,3-diamine relative to di-n-decyldimethyl ammonium chloride (DDAC) and alkyl dimethylbenzyl ammonium chloride (benzalkonium chloride, ADBAC) is between 40% and 75%.

For example, compare Test Results (LogR values) from Tests T1.CA.15.17, T1.CA.15.3, T1.CA.15.11, T1.CA.15.7, T1.CA. 15.14 shown in Table 3 or alternatively compare Test Results (LogR values) from Tests T1.CA.15.16, T1.CA.15.1, T1.CA.15.9, T1.CA. 15.6, T1.CA.15.14.

When the proportion of N-(3-aminopropyl)-N-dodecylpropane-1,3-diamine relative to di-n-decyldimethyl ammonium chloride (DDAC) and alkyl dimethylbenzyl ammonium chloride (benzalkonium chloride, ADBAC) is at or above 20% the anti-yeast efficacy as represented by the LogR values is improved by the presence of both di-n-decyldimethyl ammonium chloride (DDAC) and alkyl dimethylbenzyl ammonium chloride (benzalkonium chloride, ADBAC) as opposed to either alone and in particular when the ratio of di-n-decyldimethyl ammonium chloride (DDAC) to alkyl dimethylbenzyl ammonium chloride (benzalkonium chloride, ADBAC) is between 1:1 and 3:5 respectively.

For example, compare Test Results (LogR values) from Tests T1.CA.15.1, T1.CA.15.2, T1.CA.15.3, T1.CA. 15.4, T1.CA. 15.5 shown in Table 3.

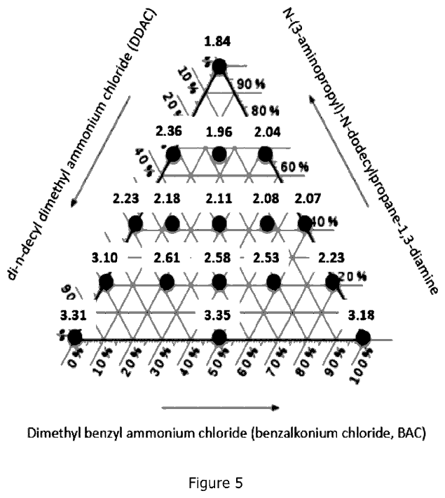

FIG. 5 shows the following trends for anti-microbial efficacy against fungus. As the proportion of N-(3-aminopropyl)-N-dodecylpropane-1,3-diamine is increased relative to di-n-decyldimethyl ammonium chloride (DDAC) and alkyl dimethylbenzyl ammonium chloride (benzalkonium chloride, ADBAC) the anti-fungal efficacy as represented by the LogR value decreases.

For example, compare Test Results (LogR values) from Tests T1.AB. 14.17, T1.AB.14.3, T1.AB.14.11, T1.AB.14.7, T1.AB.14.14 shown in Table 4 or alternatively compare Test Results (LogR values) from Tests T1.AB.14.16, T1.AB.14.1, T1.AB.14.9, T1.AB.14.6, T1.AB. 14.14.

1-23. (canceled)

24. An anti-microbial composition comprising:
(i) an active agent component comprising:
(A) a compound of formula (A) 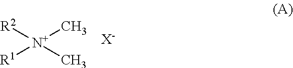
wherein R¹ and R² are independently a C₈₋₁₂ alkyl group and X⁻ is a halide anion such as chloride, bromide, fluoride, iodide or sulphonate, saccharinate, carbonate or bicarbonate;
(B) a compound of formula (B) 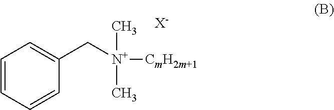
wherein m is from 8 to 18, and X⁻ is a halide anion such as chloride, bromide, fluoride, iodide or sulphonate, saccharinate, carbonate or bicarbonate; and
(C) a compound of formula (C) 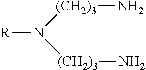
where R is an unsubstituted C₈ to C₁₈ alkyl group;
wherein:
(a) (C) is present in an amount of from 20 to 40 wt % of the active agent component; and
(b) the ratio by weight of A:B is 0.4:1 to 1:0.4; and
(ii)a polar solvent;
(iii)a chelating agent;
wherein the composition does not comprise a siloxane, silicone or polysiloxane and does not comprise a hydrophilic polymer comprising at least two of the following types of monomer:
(1) a monomer having a permanent cationic charge or a monomer that is capable of forming a cationic charge upon protonation;
(2) an acidic monomer having an anionic charge or capable of forming an anionic charge; and
(3) a neutral monomer.

25. The composition according to claim 24, wherein:
(A) is di-n-decyldimethyl ammonium chloride (DDAC); and/or
(B) is alkyl (C12-16) dimethylbenzyl ammonium chloride (benzalkonium chloride, ADBAC); and/or
(C) is N-(3-aminopropyl)-N-dodecylpropane-1,3-diamine.

26. The composition according to claim 24, wherein the ratio by weight of A:B is 3:5 to 5:3.

27. The composition according to claim 24, wherein the pH of the composition is from about 8.5 to about 11.5.

28. The composition according to claim 24, wherein the chelating agent is selected from EDTA (Ethylenediaminetetraacetic acid), Gluconate, GLDA (Glutamic acid diacetic acid), EDDS (Ethylenediamine-N,N′-disuccinic acid), DPTA (Diethylene triamine pentaacetic acid), HEDTA (Hydroxyethyl-ethylenediamine triacetic acid), MGDA (Methyl glycine diacetic acid), PDTA (1,3-propylenediaminetetraacetic acid), and EDG (Ethanoldiglycineic acid) and mixtures thereof.

29. The composition according to claim 28, wherein the chelating agent is GLDA (Glutamic acid diacetic acid).

30. The composition according to claim 24, wherein the polar solvent is selected from water, ethanol, n-propanol, isopropanol, ethylene glycol ethers, propylene glycol ethers, butyl diglycol (BDG) and dipropylene glycol methyl ether and mixtures thereof.

31. The composition according to claim 24, wherein the composition is substantially free of alcohol.

32. The composition according to claim 24, comprising a pH modifier.

33. The composition according to claim 32, wherein the pH modifier is selected from citric, sulfamic, hydrochloric, phosphoric, nitric, lactic, formic, acetic, salicylic, methanoic, glycolic and gluconic acids, sodium or potassium hydroxide, sodium carbonate, potassium carbonate, sodium bicarbonate, potassium bicarbonate, triethanolamine and monoethanolamine and mixtures thereof.

34. The composition according to claim 24 comprising a non-ionic, cationic or amphoteric surfactant.

35. The composition according to claim 34 comprising a non-ionic surfactant.

36. The composition according to claim 35 comprising an alcohol ethoxylate.

37. The composition according to claim 36, wherein the alcohol ethoxylate is a C16-18 alcohol ethoxylate.

38. A method for reducing the number of microbes on or at a surface, the method comprising applying the composition according to claim 24 to the surface.

39. The method of claim 38, wherein the method reduces and/or controls viruses on or at a surface.

40. The method of claim 38, wherein the surface is a hard surface.

41. A method for reducing the number of microbes on or at a surface and removing soil from that surface, the method comprising applying the composition according to claim 24 to the surface.

42. The method of claim 41, wherein the surface is a hard surface.

43. An anti-microbial wipe comprising a substrate and the anti-microbial composition according to claim 24.
